(19) United States
(12) Patent Application Publication
(10) Pub. No.: US 2007/0105216 A1
(43) Pub. Date: May 10, 2007

(54) Method for the controlled release of calcium from intracellular calcium stores of oocytes

(75) Inventors: Jean-Pierre Ozil, Chaville (FR); Bernadette Banrezes, Paris (FR); Daniel Huneau, Massy (FR)

Correspondence Address: FOLEY AND LARDNER LLP;SUITE 500, 3000 K STREET NW, WASHINGTON, DC, 20007, US

(73) Assignee: INSTITUT NATIONAL DE LA RECHERCHE AGRONOMIQUE

(21) Appl. No.: 11/603,238

(22) Filed: Nov. 22, 2006

Related U.S. Application Data
(62) Division of application No. 10/854,743, filed on May 27, 2004, now abandoned.

(51) Int. Cl. C12N 5/00; C12N 5/02

(52) U.S. Cl. 435/325

(57) ABSTRACT

A method for the controlled release of calcium from intracellular calcium stores of oocytes, by i) establishing an optimum profile for release of calcium from intracellular calcium stores of oocytes, and ii) applying the following cycles to the oocytes: (A) a pulse perfusion cycle which comprises a step of perfusing with one or more calcium pulse media and a step of subjecting to an electric pulse, and (B) sequences of compensating perfusion cycles which comprise a step of perfusing continuously with a calcium ion-free medium, followed by a step of perfusing with a calcium ion-containing medium; wherein the rhythms of the electric pulses and of the perfusion steps are adjusted in order to reproduce the optimum profile i).

The present invention relates to a method for the controlled release of calcium from intracellular calcium stores of oocytes or of fertilized oocytes corresponding to a profile established by virtue of a combination of pulse perfusion which comprises a perfusion step with one or more calcium media, an electric pulse step, and a step of perfusion with one or more calcium media and compensating perfusion sequences which comprise a step of continuous perfusion of a calcium ion-free medium, followed by a step of perfusion of a medium containing calcium ions.

The present invention relates to the field of assisted fertilization in humans and in animals, and concerns more particularly the step of activation of oocytes fertilized by the intracytoplasmic sperm injection (ICSI) technique.

In all mammals, at the time of fertilization, entry of the sperm into the oocyte will have two main functions:
introduction of the male haploid genome,
activation of the development which restructures the male nucleus via the oocyte cytoplasm and promotes the nucleus-cytoplasm interactions.

This second function of the sperm is related to the introduction into the oocyte of a sperm factor which triggers the intracellular release of inositol 1,4,5-triphosphate (IP3). When the IP3 binds to the IP3 receptors of the calcium channels of the endoplasmic reticulum, it increases their probability of opening as a function of the concentration of free intracellular calcium. The calcium release therefore becomes calcium-excitable. This phenomenon of calcium excitability is a characteristic of fertilization in mammals. The calcium thus released activates specific proteins which, themselves, activate specific processes, for example calcium-calmodulin complexation, kinase activation, etc. Calcium is considered to be the main metabolism activator. This reaction cycle recurs over a period of the order of several minutes for several hours. This dynamic of calcium signals triggers the initiation of embryonic development.

A calcium diagram corresponding to FIG. 2 is thus observed.

Depending on the species, 12 to 20 hours, on average, elapse between fertilization and the first cell division, during which time a set of phenomena take place, the two maternal and paternal genomes having complementary roles for subsequent development of the embryo.

This initial phase of development, called “activation”, comprises all the events between gamete fusion and formation of the parental pronuclei, i.e. cortical granule expulsion, the substantial decrease in the activity of the kinases (maturation-promoting factor (MPF) and mitogen activated protein kinase (MAPK)) involved in the unblocking of meiosis, segregation of the maternal chromosomes, reorganization of the paternal chromosomes and formation of the parental pronuclei. Without activation, the simple introduction of a set of paternal chromosomes of a somatic nucleus into an oocyte does not permit embryonic development. All these events are under the control of calcium signal dynamics.

Activation is a biological process essential to embryonic development. In this context, controlling the calcium signal dynamics makes it possible, in terms of embryo biotechnology applications, to be able to trigger, stabilize, standardize and ensure the reproducibility of the biological responses of the embryos.

In fact, the variability in the responses of the fertilized oocytes during this activation period constitutes a problem. The number of calcium signals, their frequency and their duration are very variable. It is known that the level of excitability can range from 0% to 100%, and can possibly take all the intermediate values. Thus, oocytes into which a sperm has penetrated may nevertheless be incapable of producing calcium signals and remain, despite the fertilization, at the metaphase II stage.

In fact, the variability is due to the complex functioning of the oocyte calciosome, with its compensating phenomena and to the amount of sperm factor introduced by the fertilizing sperm.

One of the objects of the present invention is to establish an optimum profile for the calcium signals of oocytes, the level of excitability of which would not be 100%, in order to ensure normal and homogeneous development of the oocytes.

In fact, the obtaining of oocytes capable of ensuring normal development of an embryo is essential to the assisted fertilization of humans and of animals. It makes it possible to limit the risks of failure in these techniques, the success rate of which still remains relatively low (approximately 16% in humans for the in vitro fertilization and embryo transfer technique).

In addition, oocytes having the potential to ensure normal development of embryos are also necessary for all embryo biotechnology in order to study fertilization and embryonic development, since the regulatory processes and factors involved in genome rearrangements are relatively unknown, and there are particularly few studies of the very early phases of oocyte development in mammals and more of this needs to be done in order to obtain a better control of in vitro fertilization processes.

In 1990, INRA proposed, in patent FR 2659347, a device for treating a large number of oocytes at the same time. This method makes it possible to periodically stimulate oocytes with calcium influxes at the plasma membrane. Specifically, by introducing oocytes into a pulsed medium comprising calcium ions, and generating an electric pulse, pores are created in the oocyte plasma membrane, which allows the calcium to penetrate into the oocyte by “electroporation”.

The method was designed on the basis of the effects of electric fields on plasma membranes. It has been shown (Zimmermann, 1982) that electric field pulses of the order of 1 to 3 kVcm⁻¹ and lasting a few μs can create micropores in the plasma membrane and can establish a direct communication between intracellular and extracellular media. It has thus been possible, for example, to cause calcium to penetrate into sea urchin oocytes by exposing them to electric field pulses in the presence of calcium ions (Rossignol et al 1983). The intensity of these influxes can be modulated by virtue of the duration of the pulse and the amplitude of the electric field. The method involves uniting a method of in vitro culturing of a batch of oocytes (or embryos) by perfusion between two electrodes and a method of perfusion with a nonconducting solution containing 0 to 20 μM calcium in which the pulses are delivered.

The presence of a very weakly conducting solution at the time of a pulse makes it possible to create an electric field between the electrodes. An excessive presence of ions decreases the “electric field” effect.

Alternating between a period of culturing and a period of perfusion makes it possible to frequently subject the oocytes to stimulations in a very well-defined ionic medium.

However, the direct calcium influxes triggered by this method in fertilized oocytes cause overexcitation of the calcium phenomena (FIG. 3) and ruin all attempts to control the activation. This method does not make it possible to obtain a very homogeneous activation of the treated oocytes, hence a considerable loss of fertilized oocytes.

To obtain 100% homogeneous responses, it is therefore necessary to be able to simultaneously stimulate oocytes which have very variable levels of excitability (the excitability may be very high or very low, going as far as a total absence due to the deficiency of sperm lacking “activating” properties).

Since it is impossible to know, a priori, the individual level of excitability of the oocytes and to provide treatments adapted for each of the oocytes, the present invention consists in implementing a series of coordinated actions in order to cause calcium signal dynamics in a standardized, parametered and reproducible manner. Since the decrease in courses of the calcium signals are identical to those which are triggered by fertilization, it is the frequency and the number thereof which come under control to produce the 100% oocyte activation and to potentiate the embryonic development.

Thus, the aim of the present invention is to synchronize and control the process of oocyte activation whatever the heterogeneity of the calcium excitability of the oocytes, so as to homogeneously graduate the biological effects triggered by the fertilization process, in particular the reorganization of all types of chromatins present in or introduced into oocytes at the time of fertilization, in order to obtain a standardized and synchronized embryonic development from a population of oocytes.

To do this, it has been noted that the methods previously used, namely the use of electric pulses and the perfusion of an external solution, with or without calcium, do not make it possible to completely regulate calcium release. In particular, either a rapid depletion of cytosolic calcium concentration or a spontaneous recommencement of calcium signals is noted.

In order to avoid these two phenomena, the applicant has noted that it is necessary to provide for a continuous bringing of the oocytes into contact, in a flow, hereinafter referred to as “perfusion”, with a calcium-containing solution and then a calcium-free solution, which makes it possible to avoid the abovementioned parasitic phenomena.

The present invention therefore relates to a method for the controlled release of calcium from intracellular calcium stores of oocytes, wherein:

i) an optimum profile for release of calcium from intracellular calcium stores of oocytes is established,

ii) cycles comprising the following are applied to the oocytes:

a pulse perfusion which comprises a step of perfusion with one or more calcium pulse media, an electric pulse step, and a step of perfusion with one or more calcium media;
sequences of compensating perfusion which comprise a step of continuous perfusion of a calcium ion-free medium, followed by a step of perfusion of a calcium ion-containing medium;
   the rhythms of the electric pulses and of the perfusion steps are adjusted in order to reproduce the optimum profile i).

The calcium release profile depends on the oocytes treated, in particular on the species. In any event, it can be determined from sampling a certain number of oocytes, the development of which it has subsequently been possible to follow.

The intracellular calcium can be assayed by methods which are known, in particular that described in Journal of Physiology (1995) Ozil and Swann, 483.2, pp. 331-346.

An example of an intracellular calcium assay technique is the measurement of the ratio of intracellular fluorescence using calcium-sensitive fluorescent probes. Such probes are, for example, Fura-2 or Indo-1.

When this technique is selected for measuring the intracellular calcium, the calcium present in the various media in which the oocytes will be placed is bound to such fluorescent probes. The binding of the calcium to the probe results in both a variation in fluorescence intensity and a shift of the excitation spectrum (Fura-2) or of the emission spectrum (Indo-1). The use of fluorescent probes such as these requires suitable measuring systems.

According to the present invention, the term “oocytes” is intended to denote fertilized and/or unfertilized oocytes.

According to the present invention, the method can be applied to unfertilized oocytes, for example an enucleated oocyte fused with a cell other than a sperm cell (haploid or diploid) for the purpose of obtaining cloned animals in order to improve their development. In the context of the use of said method according to the present invention, for enucleated oocytes into which a nucleus has been injected, it should be noted that the oocyte has an excitability level of 0%. It is not therefore necessary to inhibit the spontaneous responses since the oocytes are not excitable.

According to the present invention, the method can be applied to fertilized oocytes. According to the present invention, the term “fertilized oocyte” is intended to denote a fertilized egg derived from the entry of a sperm into an oocyte; this will involve in particular oocytes obtained by introduction of a sperm by the intracytoplasmic sperm injection (ICSI) technique.

According to the present invention, the term “intracellular calcium stores” is intended to denote, inter alia, endoplasmic reticulum and mitochondria.

According to the present invention, the term “IP3” is intended to denote inositol 1,4,5-triphosphate.

In the context of assisted reproduction, the method according to the present invention makes it possible to assist the process of oocyte activation so as to optimize parental chromosome reorganization and to promote embryonic development.

In the context of reproductive biotechnology, this method makes it possible to trigger, synchronize and optimize the embryonic development of experimental embryos in which a cell other than a sperm cell is used to obtain embryonic development.

This technology is used for assisted fertilization in humans and in animals. In fact, intracytoplasmic sperm microinjection (ICSI) is the newest of the assisted fertilization techniques. It consists in injecting a whole sperm into the cytoplasm of an oocyte. This technique is used, for example, when there are too few motile spermatozoa in the sperm for fertilization to take place. A fertilization rate which is higher than with conventional in vitro fertilization (IVF) technology is thus obtained.

The pulse perfusion comprises perfusion with one or more media containing IP3, calcium and, preferably, a potassium gluconate and, optionally, glucose.

As regards the compensating perfusions, the medium is preferably a culture medium in which the calcium ions have been replaced with other ions, in particular magnesium ions or sodium ions.

In a more detailed manner, this method comprises the following steps:
a) an optimum profile for release of calcium from intracellular calcium stores of oocytes is established;
b) the oocytes are perfused with a pulse medium 1 comprising glucose, potassium gluconate, IP3 and calcium, subjected to an electric pulse generated by an electric field, and are then perfused with a medium 2 comprising potassium gluconate, IP3 and calcium;
   The time taken for replacing the culture media totally by the pulse medium 1 should be preferably 5 seconds but no more than 20 seconds. The time taken for replacing the pulse medium 1 by the pulse medium 2 after the electrical pulse should be preferably 200 milliseconds but no more than 5 seconds;
c) the oocytes are perfused continuously in a calcium-free medium 3 and are then perfused in a calcium-containing medium 4;
d) step c) is repeated a certain number of times in order to perform sequences of compensating perfusion so as to maintain the calcium homeostasis in the oocytes;
e) steps b) to d) are repeated a certain number of times until the profile established in step a) is obtained, without any deleterious effect on the oocytes.

More particularly, according to the present invention, the IP3 concentrations of media 1 and 2, which may be identical or different, are between 2 and 100 μM. Preferably, the IP3 concentration in media 1 and 2 is identical and is 20 μM.

More particularly, according to the present invention, the calcium concentrations of media 1 and 2, which may be identical or different, are between 5 and 100 μM Preferably, the calcium concentration in media 1 and 2 is identical and is 10 μM.

More particularly, according to the present invention, medium 4 contains a calcium concentration of between 0.5 and 6 mM. Preferably, the calcium concentration in medium 4 is 1.7 mM.

More particularly, medium 3 is a medium in which all the calcium ions have been replaced with other ions, in particular sodium ions or magnesium ions. More particularly, according to the present invention, medium 3 comprises magnesium ions which replace the calcium ions, in addition to the magnesium initially present in the medium 3, at a concentration of between 0.5 and 6 mM. Preferably, the concentration of magnesium in medium 3 in addition to the magnesium initially present in said medium is 1.7 mM.

More particularly, according to the present invention, medium 3 comprises sodium ions which replace the calcium ions, in addition to the sodium initially present in medium 3, at a concentration of between 0.5 and 6 mM. Preferably, the concentration of sodium in medium 3 in addition to the sodium initially present in said medium is 1.7 mM.

Optionally, the method according to the present invention is characterized in that an additional step of putting the oocytes on hold in a medium 3 is added. This optional step can be added between step i) and step ii) or between step a) and step b).

Medium 3 is a calcium-free medium, the flow of which has the effect of greatly reducing the electrochemical calcium gradient of the oocyte and therefore of depleting the cytosolic calcium concentration. Under these conditions, the physiological process of calcium release from intracellular stores is inhibited. The process of oocyte activation is slowed down, which makes it possible to put the oocytes on hold for the period of time required to constitute a batch of oocytes. The use of a permanent flow of calcium ion-free culture medium promotes the diffusion of calcium ions from the cytosol to the outside of the cell.

As soon as the desired number of oocytes is obtained, the oocytes can be subject to step b).

The aim of step b) is, initially, to sensitize the IP3 channels by injecting the IP3 present in the pulse medium by electropermeabilization due to the electric pulse. IP3 is a small molecule which is rapidly metabolized by the oocyte. Injection thereof does not directly cause calcium release from the reticulum, but sensitizes the entire receptor population. The receptors that are already sensitized will not be further sensitized, but those which have not been sensitized will become so during the time the IP3 is present. Secondly, these steps will maintain a calcium influx necessary for the release of calcium from the internal calcium stores. Since all the calcium channels are sensitized with the IP3, it is the calcium ions which cause the opening of the channels. Maintaining the flow of calcium medium outside the oocyte containing calcium, during the release of the intracellular calcium, limits the leaking of calcium from the oocyte to the outside and promotes filling of the internal calcium stores of the oocytes.

It is imperative to completely replace the oocyte culture medium with pulse medium 1 in step b), since the strength of the current due to excess ions originating from the culture medium would destroy the oocytes. Just after the pulse, pulse medium 1 is replaced with culture medium 2, it being impossible for the oocytes to survive in pulse medium 1.

Step c) comprises, firstly, a continuous perfusion of a calcium-free medium which serves to inhibit the possible spontaneous calcium oscillations triggered by the calcium influx, firstly, by stopping the influx of IP3 and, secondly, by inhibiting the excitability. This promotes dissociation of the calcium from the IP3 receptors and limits their probability of opening.

Secondly, step c) comprises a transient perfusion, of short duration, with a calcium-rich medium in order to promote calcium ion influxes into the oocyte. This exposure time is sufficiently short so as not to cause any recommencement of spontaneous oscillations.

The repeating of step c) a certain number of times makes it possible to compensate for the leaking of calcium ions which destabilize the dynamic behavior of the calciosome.

Advantageously, according to the present invention, steps b) to d) of said method, to which the oocytes are subjected, are repeated several times, with a frequency which can vary, it being possible for the pulse duration and the electric field amplitude to be different each time.

This method makes it possible to modulate the frequency of the signal by virtue of the duration between two perfusions, and its amplitude by virtue of the duration or the voltage of the electric pulse.

This method can be controlled via a software which will make it possible to create stimulation treatments according to specific, exponential, sinusoidal equations, Fourier series, or the like.

Advantageously, according to the present invention, the release of calcium from intracellular calcium stores of oocytes is proportional to the amount of time the oocytes are exposed to medium 2. This calcium release is correlated to the time of exposure to medium 2, and more particularly to the IP3 present in medium 2.

More particularly, the longer the exposure time of the oocytes to medium 2, and therefore to the IP3, the greater the release of calcium from the intracellular calcium stores of the oocytes.

One of the aspects of the present invention is the simultaneous treatment of a set of oocytes, which will thus be obtained activated in the same physiological state.

It is extremely advantageous to be able to standardize the various phases of the oocyte treatment in order to obtain good reproducibility of the method.

Preferably, according to the present invention, the oocytes in the method are fertilized oocytes.

The present invention therefore relates to a dynamic system, which functions continuously and which can be automated, implemented by a device which allows the automatic succession of the perfusion and stimulation steps and the acquisition of the stimulation parameters.

This device ensures that the oocytes are immobilized during the various treatment phases. Thus, according to another aspect, the present invention also relates to an oocyte culturing device for implementing the method according to the present invention, which makes it possible to align the oocytes and synchronize the controlled release of calcium from intracellular calcium stores of the oocytes, and which consists of a chamber, intended to accept the oocytes, the lower part of the chamber comprising one or more orifices, the geometry of which is such that it does not allow the oocytes to pass through, and the chamber comprising at least one liquid injection pipe.

Preferably, according to the present invention, the oocytes in the device are fertilized oocytes.

The present invention will be understood more clearly from the further description which follows, which refers to examples of controlled release of calcium from oocytes according to the present invention.

It goes without saying, however, that these examples are given only by way of illustration of the subject of the invention, of which they could in no way constitute a limitation.

### FIGURE LEGEND

FIG. 1a: Physiological responses of a nonfertilized oocyte after a pre-pulse perfusion, an electric pulse and a post-pulse perfusion.

FIG. 1b: Physiological responses of a nonfertilized oocyte after a pre-pulse perfusion without IP3, an electric pulse and a post-pulse perfusion without IP3.

FIG. 2: Physiological responses of an oocyte fertilized by a sperm, without the action of the method of the present invention.

FIG. 3: Spontaneous response in the form of a series of calcium oscillations triggered by an influx of calcium ions into a fertilized oocyte.

FIG. 4: Physiological responses of an oocyte fertilized by a sperm, according to the ICSI method, with the action of the method of the present invention, without steps c) and d), corresponding to calcium reloading of the oocyte, but with perfusion with medium 3.

FIG. 5: Physiological responses of an oocyte fertilized by a sperm, according to the ICSI method, with the action of the method of the present invention, with, in steps c) and d), a perfusion of the oocytes with the calcium-rich medium for 30 seconds every 30 seconds, and then 5 seconds every 55 seconds.

FIG. 6a: Physiological responses of an oocyte fertilized by a sperm, according to the ICSI method, with the action of the method of the present invention (stimulation frequency=8 minutes).

FIG. 6b: Physiological responses of an oocyte fertilized by a sperm, according to the ICSI method, with the action of the method of the present invention (stimulation frequency=32 minutes).

### EXAMPLES

Controlled Release of Calcium from Mouse Oocytes Fertilized by ICSI:

### 1. OBTAINING FERTILIZED OOCYTES

After introduction of a sperm into an oocyte by the ICSI (IntraCytoplasmic Sperm Injection) technique, the oocytes are placed at 37° C. in a permanent flow of calcium ion-free culture medium (M16 Ca2+ free). The strength of this flow can range between 2 and 10 μL/second depending on the geometry of the perfusion chamber.

The effect of the decrease in the electrochemical gradient outside the cell is to deplete the cytosolic calcium concentration. Under these conditions, the physiological process of calcium release from the intracellular stores is inhibited. The process of oocyte activation is slowed down, which makes it possible to put the oocytes on hold for the period of time required to constitute a batch of experimental oocytes. The use of a permanent flow of calcium ion-free culture medium promotes diffusion of the calcium ions from the cytosol to the outside of the cell.

As soon as the desired number of experimental oocytes is attained, the oocytes are subjected to the following action.

### 2. PRE-PULSE PERFUSION

The treatment begins with the rapid replacement of the culture medium with a solution made up of: glucose (50 g/liter), potassium gluconate (100 μM), CaCl₂ (10 μM) and IP3 (20 μM). The strength of the perfusion flow can range from 10 to 15 μL/sec and its duration can range from 5 seconds to 20 seconds depending on the geometry of the chamber. The effect of this perfusion is to decrease the ionic strength of the external medium below the zona pellucida of the oocytes so as to limit the strength of the electric currents at the time of electropermeabilization of the plasma membrane. The composition of this medium corresponds to five aims.

Firstly, to very rapidly replace all the molecular species of the culture medium with a restricted number of molecules so as to limit the diversity and the strength of the transmembrane fluxes of ions or of molecules at the time of electropermeabilization of the plasma membrane. The duration of exposure of the oocytes in this depleted medium should be as short as possible so as to limit the deleterious effects induced by the disturbances of the cell's electrochemical potential. The minimum duration is related to the rate of molecular diffusion across the zona pellucida. This diffusion coefficient is for the moment unknown. We consider, however, that a minimum duration of 5 seconds under our experimental conditions is necessary and sufficient to ensure evacuation of the undesirable ions and molecules in the perivitelline (below the zona pellucida) which surrounds the cell.

Secondly, the presence of glucose (40 to 60 g/L) provides the isotonicity of the medium in a medium which has a low ionic strength.

Thirdly, the presence of K+ ions (100 μM at least in the form of potassium gluconate) makes it possible to create a low-amplitude ion current at the time of electropermeabilization. This K+ current is biologically neutral. Its role is to facilitate the phenomenon of plasma membrane permeabilization.

Fourthly, the presence of IP3 in the perfusion medium makes it possible to introduce this small molecule (IP3) into the cell, by electropermeabilization. The IP3 sensitizes the IP3-dependent calcium channel located in the membrane of the endoplasmic reticulum. The IP3 does not directly cause the release of calcium from the reticulum, but sensitizes the entire receptor population. The receptors which are already sensitized by the contribution of the sperm will not be further sensitized, but those which are not sensitized will become so during the time the IP3 is present.

Fifthly, the presence of calcium ions (at least 10 μM) makes it possible to create a calcium influx into the cell so as to increase the cytosolic calcium concentration above a threshold value for which the IP3 channels open and release the calcium from the stores. In this case, the calcium influx is the trigger for the physiological signal. It is the reason for its presence. The relative proportion of all these components in the perfusion medium can be optimized as a function of the developmental potential of the embryos thus treated, but also as a function of the species to which the oocytes belong. Once perfused, the oocytes are subjected to the following action.

### 3. ELECTRIC PULSE

A high-amplitude electric pulse (from 0.5 to 1.5 KVcm⁻¹) for 300 μs at a frequency of 10 kHz is triggered. This pulse causes permeabilization of the plasma membrane.

### 4. POST-PULSE PERFUSION

Within milliseconds following the electric pulse, the oocytes are immersed in a medium made up of potassium gluconate (K+) (120 mM) containing 20 μM of IP3 and 10 μM of calcium.

The composition of this medium corresponds to 3 aims.

Firstly, this medium makes it possible to maintain the permeabilized membrane state due to the action of the potassium gluconate, the composition and the concentration of which mimics the intracellular medium.

Secondly, this medium makes it possible to maintain an extracellular calcium ion concentration greater than the cytosolic concentration during the calcium signal, which can reach 3 μM, so as to limit calcium effluxes during this signal phase. The absence of calcium ions outside the oocyte promotes a calcium efflux and therefore shortens the duration of the signal. The presence of calcium ions in the external medium limits and compensates for these deleterious effects. This extracellular concentration can reach 100 μM. Beyond this value, the calcium ions promote restoration of selective permeability of the membrane.

Thirdly, this medium makes it possible to maintain an extracellular IP3 concentration of 20 μM. The duration of IP3 presence in the extracellular medium promotes as complete an emptying of calcium from the intracellular stores as possible, which also promotes termination of the signal via a mechanism as yet unexplained but completely reproducible, which is used in the present case. The duration of this phase can range between 30 and 60 seconds. This action makes it possible to maintain the oocyte membrane in a permeabilized state in the presence of calcium and of IP3.

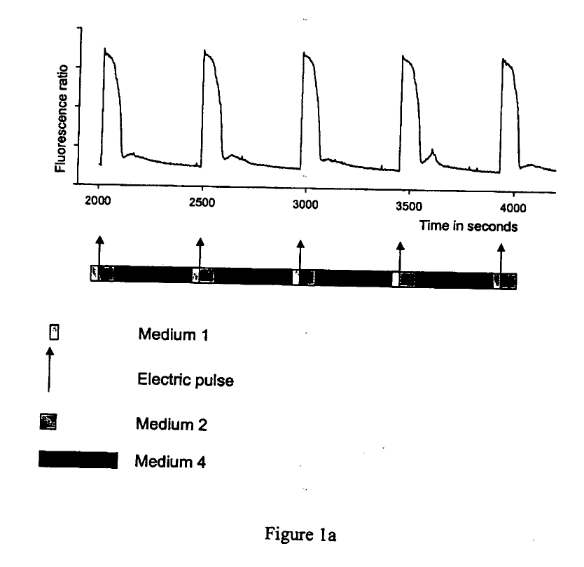

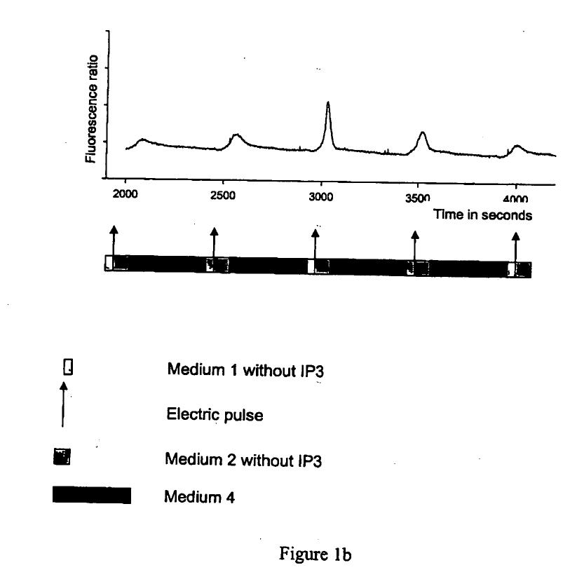

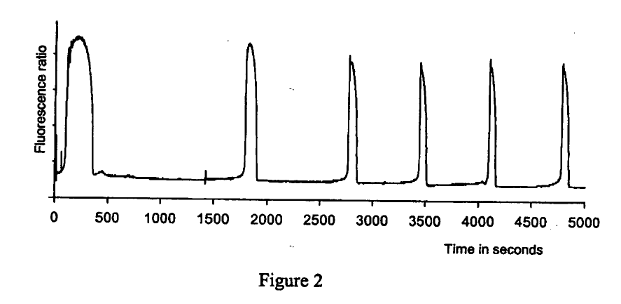

FIG. 1a shows that, in the case of a nonfertilized oocyte, this action causes a calcium signal comparable to that which can be observed during fertilization, as shown in FIG. 2.

However, if the nonfertilized oocytes are perfused in a medium containing no IP3, the decrease in course of the signal is altered, as shown in FIG. 1b.

This example shows that physiological responses are obtained in nonfertilized oocytes in which the calcium system has not therefore been sensitized by fertilization.

In the case of fertilized oocytes, into which a sperm has been introduced, this action therefore triggers all the more a physiological signal, as shown in FIG. 2. In fact, the fertilized oocyte is sensitized by the fertilization and by the action of the combination of perfusions with media containing IP3 and calcium and by the electric pulses.

The following action is aimed at inhibiting spontaneous calcium signals due to the self-amplifying phenomenon referred to as CICR (Calcium Induced Calcium Release) in order to ensure control of the number and of the frequency of the signals.

### 5. INHIBITION OF SPONTANEOUS CALCIUM SIGNALS

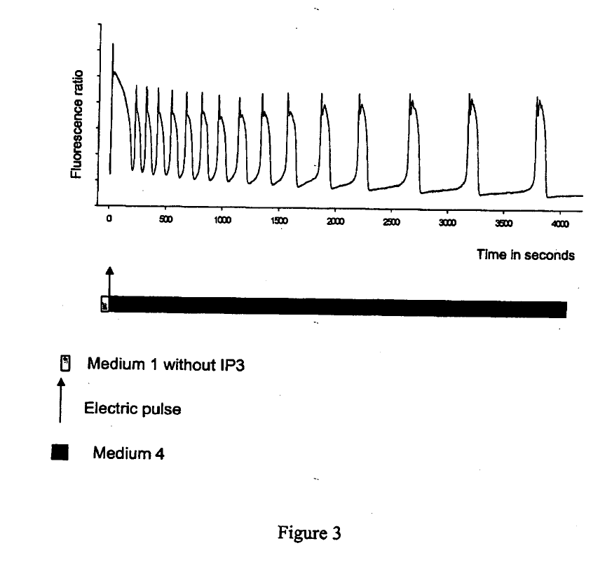

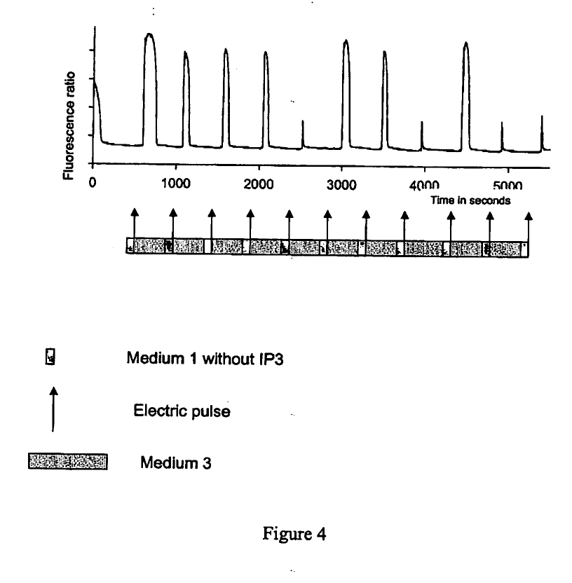

Once the calcium signal is over, it becomes necessary to maintain the cytosolic calcium concentration below the threshold from which the signals trigger spontaneously. FIG. 3 shows the spontaneous response in the form of a series of calcium oscillations triggered by an influx of calcium ions in a fertilized oocyte in which the calcium signals have not been inhibited. This operation is dynamic by nature since the oocyte possesses many pumps and channels which maintain an equilibrium between the endoplasmic reticulum, cytosolic and extracellular concentrations. Permanently maintaining the oocytes in a flow of calcium-free culture medium causes an imbalance between these three compartments. The cytosolic calcium concentration decreases; this is the desired effect. However, when this action is prolonged, the oocyte increasingly loses calcium. Over time, this loss of load causes considerable inhibition of the calcium excitability, which becomes resistant to any stimulation. As can be seen in FIG. 4, irregularities in the oocyte's responses, which are due to the loss of calcium load, are observed. The obtaining of stable responses becomes uncertain.

### 6. LIMITING CALCIUM LOAD LOSSES

In order to limit this phenomenon of loss of calcium load and to maintain the calcium excitability, the oocytes were perfused for 5 seconds every 55 seconds with a culture medium containing 1.7 mM of calcium (M16 medium). The presence of calcium in the external medium for 5 seconds every minute makes it possible to limit the calcium load losses while inhibiting the spontaneous signals.

The combining of these two types of perfusions (without calcium and with calcium) makes it possible to limit this phenomenon of loss of calcium load. A perfusion with a calcium-free medium makes it possible to reduce the cytosolic level and a perfusion with medium containing calcium makes it possible to limit the calcium load losses.

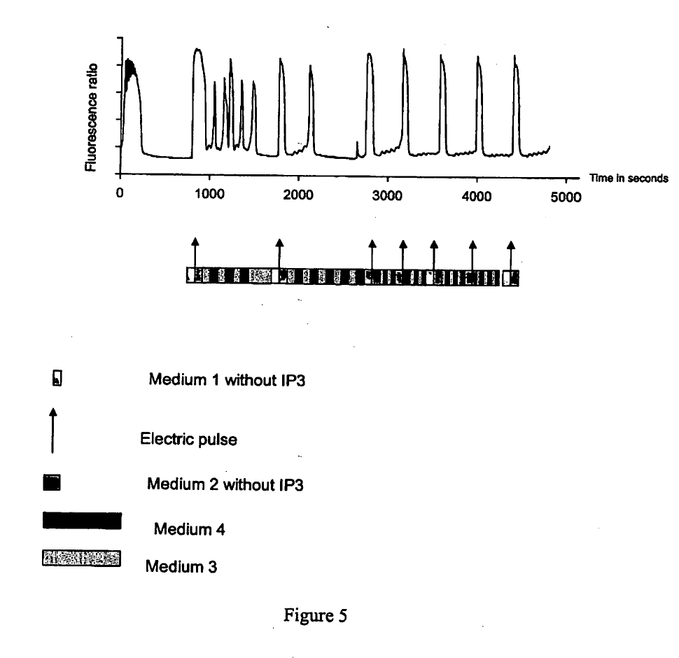

These two phases (1 min) are repeated a certain number of times and make it possible to define a time interval between two calcium signals. In principle, the desired effect could be obtained by adjusting an extracellular calcium concentration corresponding to the cytosolic threshold value for which there is no CICR activity. This threshold value is unknown and probably difficult to determine for an oocyte population On the other hand, alternating perfusions with and without calcium makes it possible very rapidly to find a dynamic equilibrium between calcium entering and calcium leaving. In the case of the mouse oocytes, the values of 55 seconds and 5 seconds are durations which correspond to the targeted aim. FIG. 5 shows that it is possible to vary the time interval between two stimulations at will, without causing desensitization or spontaneous signals.

### 7. CONCLUSION

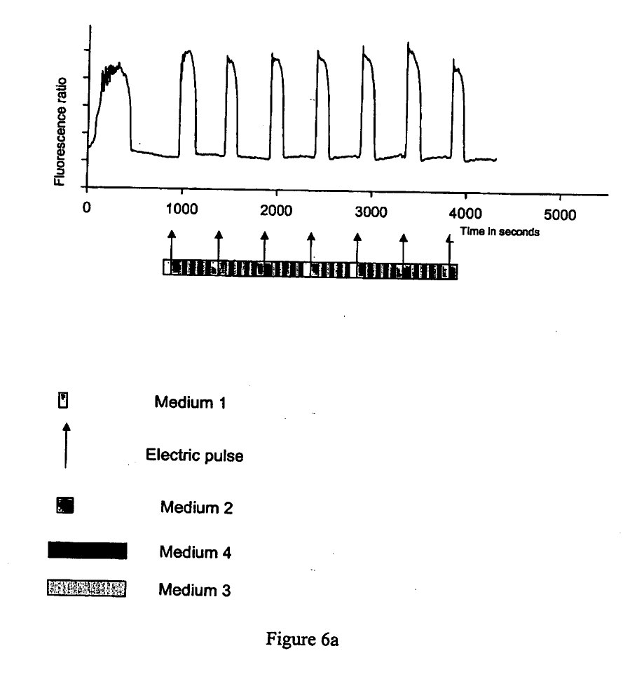

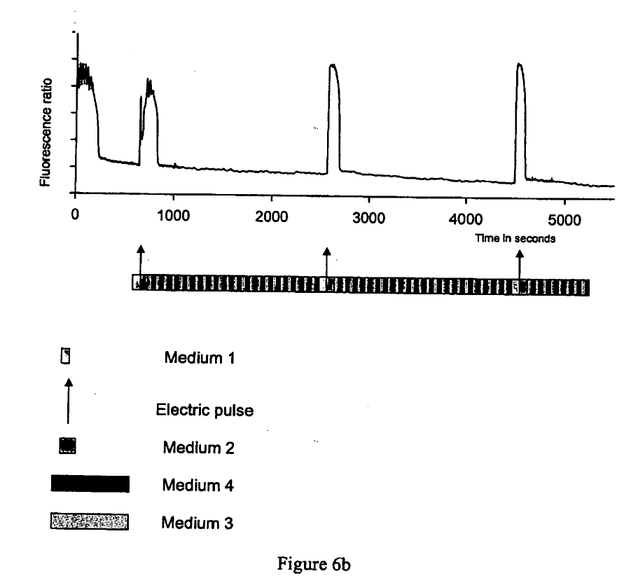

In the end, a profile for release of calcium from intracellular stores of fertilized oocytes, such as those presented in FIG. 6a (with a frequency of stimulation every 8 minutes) and in FIG. 6b (with a frequency of stimulation every 32 minutes), is obtained.

1-21. (canceled)

22. A method for the controlled release of calcium from intracellular stores of fertilized oocytes containing calcium, comprising:
i) establishing an optimum profile for release of calcium from intracellular calcium stores of fertilized oocytes,
ii) perfusing with one or more pulse media comprising calcium, iii) subjecting to an electric pulse,
iv) perfusing continuously with a calcium ion-free medium,
v) perfusing with a calcium ion-containing medium,
wherein steps iv) to v) are repeated in order to maintain calcium homeostasis in the fertilized oocytes and step ii) to v) are repeated or not until the profile established in step i) is obtained.

23. The method as claimed in claim 22, comprising:
i) establishing an optimum profile for release of calcium from intracellular calcium stores of fertilized oocytes,
ii) perfusing with a first pulse medium (medium 1) comprising glucose, potassium gluconate, inositol 1,4,5-triphosphate (IP3) and calcium,
iii) subjecting to an electric pulse,
ii) perfusing with a second pulse medium (medium 2) comprising potassium gluconate, IP3 and calcium,
iv) perfusing continuously with a calcium ion-free medium (medium 3),
v) perfusing with a calcium ion-containing medium (medium 4),
wherein steps iv) to v) are repeated in order to maintain calcium homeostasis in the fertilized oocytes.

24. The method as claimed in claim 23, wherein the IP3 concentrations of media 1 and 2, which may be identical or different, are between 2 and 100 μM.

25. The method as claimed in claim 24, wherein media 1 and 2 contain an IP3 concentration of 20 μM.

26. The method as claimed in claim 23, wherein the calcium concentrations of media 1 and 2, which may be identical or different, are between 5 and 100 μM.

27. The method as claimed in claim 26, wherein media 1 and 2 contain a calcium concentration of 10 μM.

28. The method as claimed in claim 23, wherein medium 4 contains a calcium concentration of between 0.5 and 6 mM.

29. The method as claimed in claim 28, wherein medium 4 contains a calcium concentration of 1.7 mM.

30. The method as claimed in claim 23, wherein the calcium ions of medium 3 have been replaced with other ions at equal concentration.

31. The method as claimed in claim 23, wherein medium 3 contains magnesium ions which replace calcium ions (Mg/Ca), if magnesium ions are initially present in medium 3 (Mg3), said Mg/Ca, being in addition of said Mg3, at a concentration of between 0.5 and 6 mM.

32. The method as claimed in claim 23, wherein medium 3 contains magnesium ions which replace calcium ions (Na/Ca), if sodium ions are initially present in medium 3 (Na3), said Na/Ca, being in addition of said Na3, at a concentration of between 0.5 and 6 mM.

33. The method as claimed in claim 31, wherein medium 3 contains magnesium ions which replace calcium ions (Mg/Ca), if magnesium ions are initially present in medium 3 (Mg3), said Mg/Ca, being in addition of said Mg3, at a concentration of 1.7 mM.

34. The method as claimed in claim 32, wherein medium 3 contains magnesium ions which replace calcium ions (Na/Ca), if sodium ions are initially present in medium 3 (Na3), said Na/Ca, being in addition of said Na3, at a concentration of 1.7 mM.

35. The method as claimed in claim 22, wherein a step of putting the fertilized oocytes in medium 3 for the period of time required to constitute a batch of fertilized oocytes is added.

36. The method as claimed in claim 35, wherein the additional step is added between step i) and step ii) or between step iii) and step iv).

37. The method as claimed in claim 23, wherein steps ii) to v) to which the fertilized oocytes are subjected are repeated several times with a frequency which vary or not, the duration and the amplitude of the electric field pulse to be identical or different each time.

38. The method as claimed in claim 23, wherein the release of calcium from intracellular calcium stores of fertilized oocytes is proportional to the amount of time the fertilized oocytes are contacted to medium 2.

39. The method as claimed in claim 22, wherein the method is applied simultaneously to a set of fertilized oocytes, which will thus be activated in the same physiological state.

40. The method as claimed in claim 23, wherein the repetition of steps ii) to v) does not involve any desensitization of calcium signal response.

41. The method as claimed in claim 30, wherein the calcium ions of medium 3 have been replaced with magnesium ions or sodium ions.
